(19) United States
(12) Plant Patent Application Publication
(10) Pub. No.: US 2002/0092062 P1
(43) Pub. Date: Jul. 11, 2002

(54) Chrysanthemum named Resolute

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/757,641

(22) Filed: Jan. 11, 2001

(51) Int. Cl. A01H 5/00

(52) U.S. Cl. PLT/288

(57) ABSTRACT

A Chrysanthemum plant named Resolute characterized by its large sized blooms with white ray-florets and green disc florets.

### RELATED APPLICATIONS

The present application is related to the following co-pending applications: Application no. Filing Date Title

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from an open pollination among groups of chrysanthemum cultivars maintained under the control of the inventor for breeding purposes. The new and distinct cultivar was discovered and selected as one flowering plant by Rob Noodelijk in a controlled environment (greenhouse) in Rijsenhout Holland in September 1998. The first act of asexual reproduction of Resolute was accomplished when vegetative cuttings were taken from the initial selection in November 1998 in a controlled environment in Rijsenhout Holland.

### SUMMARY OF THE INVENTION

The present invention is a new and distinct variety of chrysanthemum bearing large sized blooms with white ray-florets and green disc florets (only visible when mature)

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

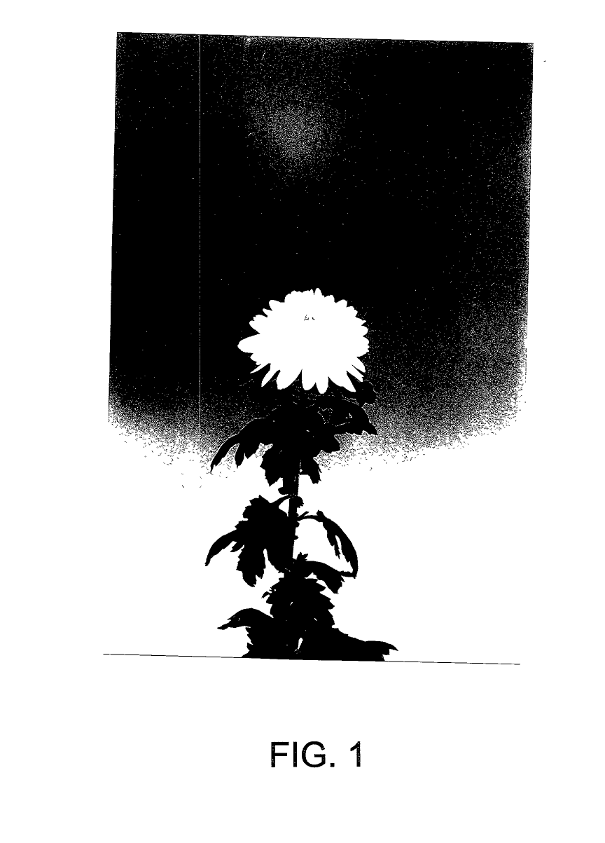

FIG. 2 shows 2 stages of the bloom.

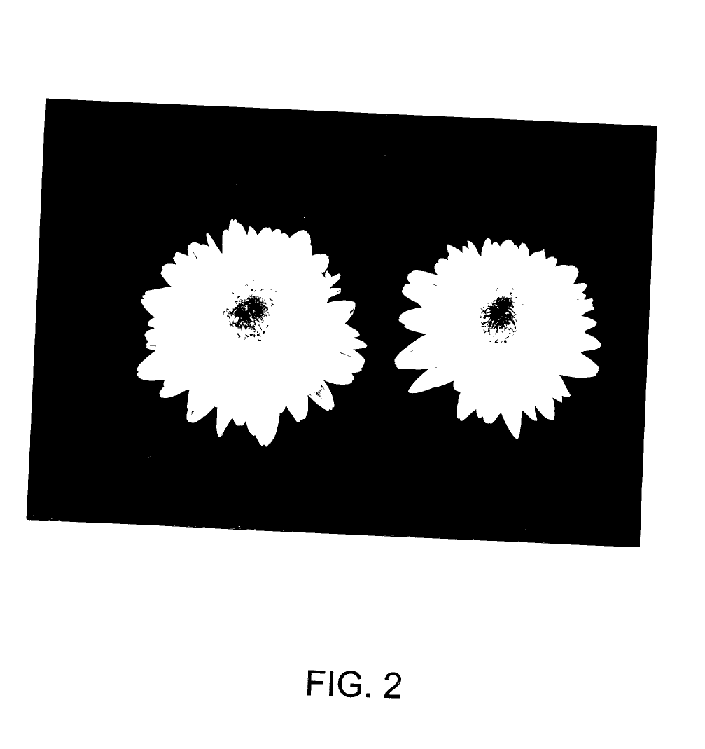

FIG. 3 shows the foliage of the cultivar.

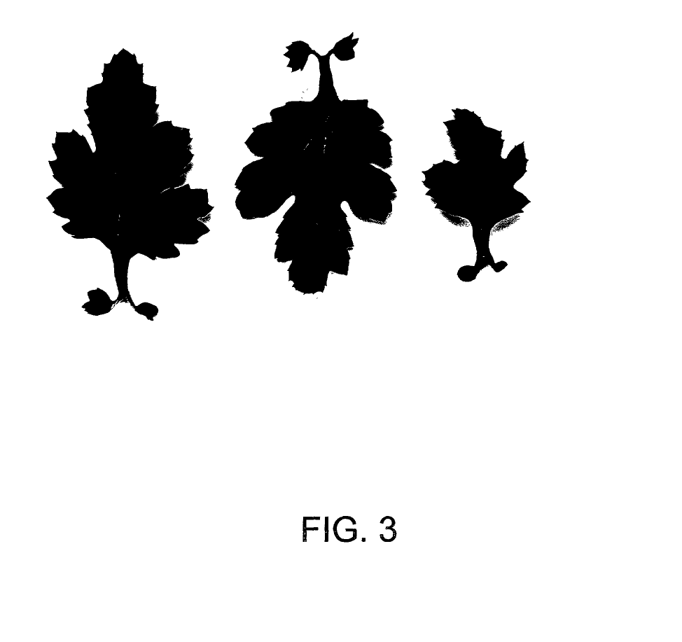

### DESCRIPTION OF THE INVENTION

This new variety of chrysanthemum is of the botanical classification Dendranthema Grandiflora. The observations and measurements were gathered from plants grown in a greenhouse in Rijsenhout Holland in a photo-periodic controlled crop. The photo-periodic response time in this crop was 50 days. The variety has been tested as a disbudded variety only. The description of the variety refers to this. This new variety produces large sized blooms with white ray-florets and green disc-florets blooming on the plant for 21 days. This new variety of chrysanthemum has been found to retain its distinctive characteristics throughout successive propagations however the phenotype may vary significantly with variations in environment such as light intensity and temperature. To show the phenotype as described Resolute may be planted without assimilation lightning between week 50 and 40 under greenhouse conditions in Holland. With assimilation lightning (minimum level 2500 lux) it may be planted year round under greenhouse conditions in Holland.

The following is a description of the plant and characteristics that distinguish Resolute as a new and distinct variety.

The color designations are taken from the plant itself. Accordingly, any discrepancies between the color designations and the colors depicted in the photographs are due to photographic tolerances.

Botanical description of CULTIVAR Resolute:

Bud:

Size.—Not applicable.

Outside color.—Not applicable.

Bloom:

Size.—Large.

Fully expanded.—9.5 cm.

Borne.—Not applicable.

Number of blooms per single stem.—Not applicable.

Peduncle length.—Not applicable.

Peduncle lateral shoot attachment.—Not applicable.

Peduncle lateral shoot angle.—Not applicable.

Form.—Decorative.

Performance (blooming period).—21 days.

Color:

Center of the flower (disc-florets only visible when mature).—Immature yellow-green 145A Mature (disc-florets)yellow-green 146A.

Color of upper surface of the majority of the ray-florets.—White 155A.

Color of the lower surface of the majority of the ray-florets.—White 155A.

Tonality from distance.—A large white decorative bloom.

Discoloration.—None.

Ray florets:

Texture.—Upper and under side smooth.

Number.—170-180.

Cross-section.—Concave.

Longitudinal axis of majority.—Straight to incurved.

Shape of tip.—Rounded.

Fragrance.—Typical chrysanthemum.

Disc diameter (when visible).—0.7 cm.

Reproductive Organs:

Stamen (present in disc florets only).—Yellow-green, thin, +/−3 mm in length.

Pollen.—Not present.

Styles (present in both ray and disc florets).—Yellow-green, thin.

Style length.—+/−4 mm.

Stigmas.—Yellow-green.

Stigma Width.—+/−1 mm.

Ovaries.—Enclosed in calyx.

Plant:

Form.—Spray mum meant for erect culture. Herbaceous.

Growth.—Medium.

Height.—100 cm.

Stem color.—Green 137A.

Stem strength.—Strong.

Stem brittleness.—Present.

Stem anthocyanin coloration.—Absent.

Internodes.—2 cm.

Flowering Response (photo-periodic controlled crop, no natural growing).—50 days.

Foliage:

Color.—Upper side green 139A under side green 138A.

Size.—large; length +/−9 cm, width +/6 cm.

Quantity (number per stem) 30-32.

Shape.—Egg shaped.

Texture.—Fleshy.

Ribs and veins.—Ribs and veins well developed.

Edge.—Crenated.

Shape of base of sinus between lateral lobes.—Acute.

Margin of sinus between lateral lobes.—Converging.

1. A new and distinct variety of chrysanthemum plant as described and illustrated.
